(19) United States
(12) Patent Application Publication
(10) Pub. No.: US 2023/0255482 A1
(43) Pub. Date: Aug. 17, 2023

(54) PORTABLE INTELLIGENT MULTI-FUNCTION PHOROPTER

(71) Applicant: Wen-Jung Lo, Taipei (TW)

(72) Inventor: Wen-Jung Lo, Taipei (TW)

(21) Appl. No.: 17/672,551

(22) Filed: Feb. 15, 2022

(51) Int. Cl. A61B 3/18; A61B 3/028; A61B 3/032; A61B 3/04; A61B 3/00; A61B 3/036

(52) U.S. Cl. CPC A61B 3/18; A61B 3/0285; A61B 3/0325; A61B 3/04; A61B 3/005; A61B 3/0075; A61B 3/036

(57) ABSTRACT

A portable intelligent multi-function phoropter is disclosed. The phoropter comprises a casing, a plurality of control elements, two adjustable eyepiece modules, a plurality of micro motors, a liquid crystal display module, a control module and a power supply module. The casing of the present invention is small so that the overall volume is small, and it is convenient to carry and use. The present invention has many test patterns, so different vision tests can be performed. With the combination of convex lens and concave lens and the application of existing electronic components, the overall price is reduced. The invention satisfies the market demand for the phoropter.

### FIELD OF THE INVENTION

The present invention relates to a phoropter. More particularly, the present invention relates to a fully functional portable intelligent multi-function phoropter.

### BACKGROUND OF THE INVENTION

As all know, the traditional phoropter is a desktop device, which is large in size and heavy in weight. Once installed, it is inconvenient to move. Therefore, when people need to use a phoropter, they must go to a specific place, such as an eye clinic or an eyeglass shop, to know the condition of their eyes. Institutions with testing vision needs, such as school health centers, require large sums of money and space for installation. For parents who are concerned about their children's eye health, even if they have to go to the eye clinic often, they will not prepare a phoropter at home for backup. If the size of the phoropter is reduced and the price is lower, it will be a boon for the aforementioned institutions or individuals. Preferably, the phoropter is multi-functional. For example, the phoropter can perform visual acuity, color blindness, astigmatism and red-green tests at the same time.

### SUMMARY OF THE INVENTION

This paragraph extracts and compiles some features of the present invention; other features will be disclosed in the follow-up paragraphs. It is intended to cover various modifications and similar arrangements included within the spirit and scope of the appended claims.

In order to fulfill the aforementioned requirements, a portable intelligent multi-function phoropter is disclosed. It comprises: a plurality of control elements, installed on the casing with external portion exposed; two adjustable eyepiece modules, movably installed on a first side of the casing; a plurality of micro motors, wherein each micro motor interacts with one of the adjustable eyepiece modules and drives to adjust the corresponding adjustable eyepiece module, so as to change the focus location for light beams emitting from the inside of the casing to the outside and a spacing between two adjustable eyepiece modules; a liquid crystal display module, installed on a second side opposite the first side of the casing and inside the casing, projecting images to the direction of the two adjustable eyepiece modules; a control module, installed in the casing and signally connected to the control elements, the micro motors and the liquid crystal display module, executing following operations: making the liquid crystal display module present an operation interface; executing a default program according to the content of the operation interface and receiving control signals from the control elements to decide execute commands; making the liquid crystal display module present a designated test pattern according to a first execute command; driving the micro motors according to a second execute command; determining a test result according to a third execute command; and recording the test result and outputting the test result according to a fourth execute command; and a power supply module, installed in the casing and electrically connected to the micro motors, the liquid crystal display module and the control module, providing power required for operation to the electrically connected components.

According to the present invention, the control elements may further comprise at least one function button and a directional key to input signals to the control module.

According to the present invention, a first adjustable eyepiece module may comprise: a first convex lens, fixed on a first movable board; and a first concave lens, fixed on a second movable board, wherein the second movable board has a second gear rack installed thereon and is movably placed on the first movable board, and the second gear rack moves the second movable board parallel to an optical axis of the first concave lens by the rotation of a gear mounted to one of the micro motors. The first movable board has a first gear rack installed thereon, and the first gear rack moves the first movable board parallel to a direction vertical to the moving direction of the second movable board by the rotation of a gear mounted to one of the micro motors.

According to the present invention, a second adjustable eyepiece module may comprise: a second convex lens, fixed on one side of the casing and inside the casing; and a second concave lens, fixed on a third movable board. The third movable board has a third gear rack installed thereon and is movably placed on the side of the casing and inside the casing, and the third gear rack moves the third movable board parallel to an optical axis of the second concave lens by the rotation of a gear mounted to one of the micro motors.

According to the present invention, the power supply module may further comprise: a USB socket, receiving external power and transmitting information; a secondary battery; a charge and discharge control chip, electrically connected to the USB socket and the secondary battery, storing the external power received by the USB socket to the secondary battery, and outputting the power in the secondary battery; and a power management chip, transforming the voltage of the power outputted from the charge and discharge control chip to a plurality of voltage values, providing to electrically connected elements, respectively.

According to the present invention, the control module further comprises: a flash memory module, storing the test result, codes and code-related data; a synchronous dynamic random access memory module; a graphics processor, signally connected to the liquid crystal display module to control the image presented by the liquid crystal display module; a wireless communication module; and a controller, signally connected to the control elements, the USB socket, the synchronous dynamic random access memory module, the flash memory module, the graphics processor, the wireless communication module and the micro motors and electrically connected to the power management chip, executing the default program to execute the operations, temporarily storing part of the codes and called code-related data in the synchronous dynamic random access memory module, and transmitting the stored test result to a receiving device wirelessly connected through the wireless communication module.

Preferably, the wireless communication module is a Bluetooth module or a Wi-Fi module.

Preferably, the test pattern is complete or part of Tumbling E Chart, Landolt C Chart, Color Blindness Test pattern, Astigmatism Test pattern or Duochrome Test pattern.

Preferably, steps for the third execute command to determine the test result comprises: recording the control element pressed by the user and an operation mode thereof according to the complete or the part of chart or pattern presented; comparing the complete or the part of chart or pattern presented, the control element pressed and the operation mode of the control element pressed with a database storing complete or each part of the chart or pattern, corresponding control elements, the operation modes of the corresponding control elements, and corresponding eye status descriptions; and selecting the eye status description corresponding to the exact comparison result as the test result.

According to the present invention, the adjustable eyepiece module may change the focus location for light beams emitting from the inside of the casing to the outside, simulating an actual distance of 20 feet to 50 feet required for eye optometry into a distance of about 10 cm.

According to the present invention, the test pattern is a simulated image of a specific object seen through a spectacle lens with a specific eyeglass prescription. Simultaneously presenting the simulated images of the specific object each seen through a spectacle lens with a specific eyeglass prescription simulates the specific object seen through the spectacle lenses superimposed.

It can be seen from the foregoing description that the casing of the present invention is small so that the overall volume is small, and it is convenient to carry and use; the present invention has many test patterns, so different vision tests can be performed; and with the combination of convex lens and concave lens and the application of existing electronic components, the overall price is reduced. Thus, the invention satisfies the market demand for the phoropter.

### BRIEF DESCRIPTION OF THE DRAWINGS

FIG. 1 is a schematic diagram of a portable intelligent multi-function phoropter according to an embodiment of the present invention.

FIG. 2 is an explosion schematic diagram of the portable intelligent multi-function phoropter.

FIG. 3 illustrates and compares structural differences between a first adjustable eyepiece module and a second adjustable eyepiece module in a direction perpendicular to a first side.

FIG. 4 shows the whole structures of the first adjustable eyepiece module and the second adjustable eyepiece module in a direction parallel to the first side.

FIG. 5 is a block diagram of a liquid crystal display module, a control module, a power supply module and micro motors.

FIG. 6 shows a Tumbling E Chart and a portion of the Tumbling E Chart presented on the liquid crystal display module.

FIG. 7 shows a Color Blindness Test pattern and a portion of the Color Blindness Test pattern presented on the liquid crystal display module.

FIG. 8 shows a complete Astigmatism Test pattern and a diagram presented on the liquid crystal display module.

FIG. 9 shows a type of Duochrome Test pattern presented on the liquid crystal display module.

FIG. 10 shows a table of test results.

FIG. 11 illustrates a portable intelligent multi-function phoropter installed on a platform which is connected to a headgear.

### DETAILED DESCRIPTION OF THE PREFERRED EMBODIMENTS

The present invention will now be described more specifically with reference to the following embodiments.

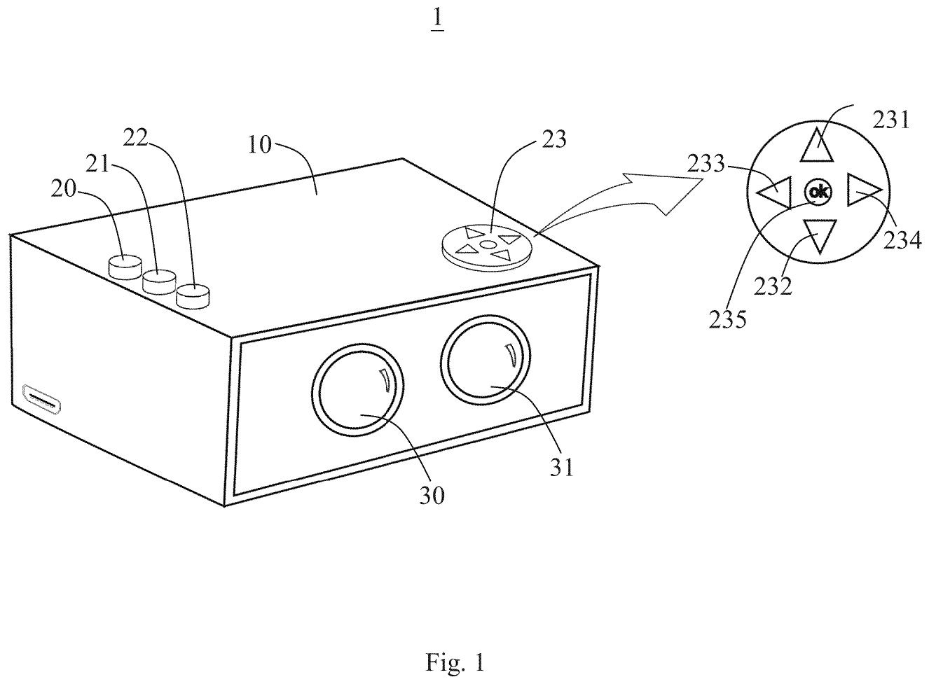

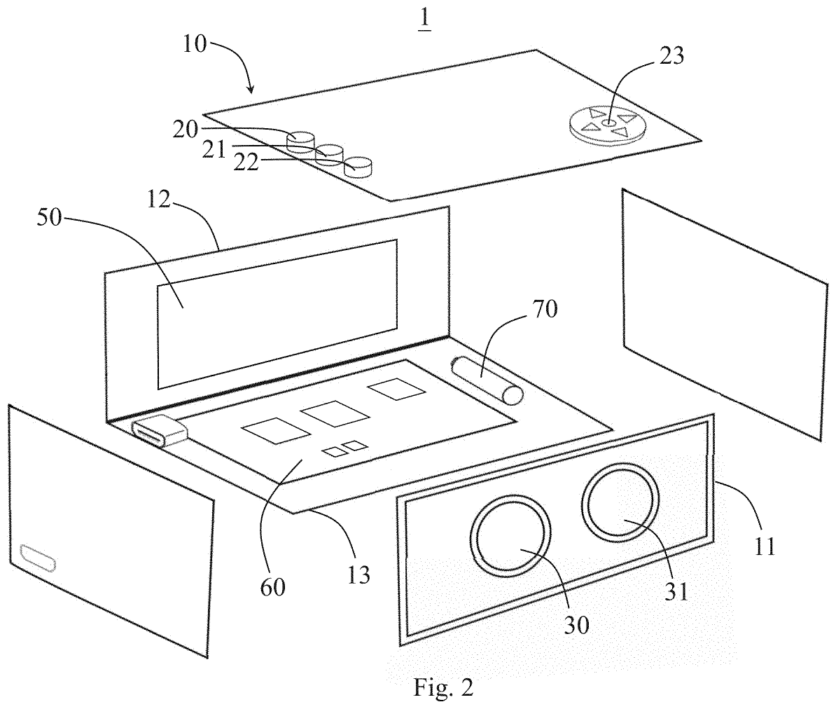

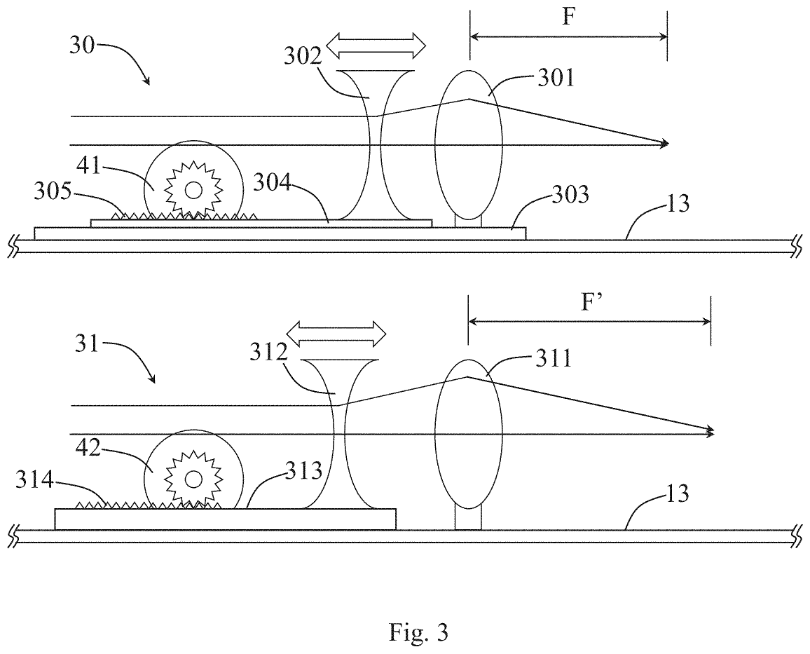

Please refer to FIG. 1 and FIG. 2. FIG. 1 is a schematic diagram of a portable intelligent multi-function phoropter 1 according to an embodiment of the present invention. FIG. 2 is an explosion schematic diagram of the portable intelligent multi-function phoropter 1. The portable intelligent multi-function phoropter 1 includes a casing 10, a number of control elements, (In the present embodiment, there are a first function button 20, a second function button 21, a third function button 22 and a directional key 23, but it is not limited thereto in practice.), a first adjustable eyepiece module 30, a second adjustable eyepiece module 31, a number of micro motors, (according to the spirit of the present invention, there should be at least 3 micro motors; a first micro motor 40, a second micro motor 41 and a third micro motor 42 are used for example in the present embodiment. See FIG. 3 to FIG. 5 for details.) a liquid crystal display module 50, a control module 60 and a power supply module 70. Types and functions of the aforementioned technical elements and the operation method and utility of the portable intelligent multi-function phoropter 1 will be illustrated with the relevant diagrams below.

The casing 10 is an element to protect internal electronic and mechanical materials in the portable intelligent multi-function phoropter 1 and convenient for testers to hold. Viewing from the outside, the casing 10 has several openings, so that the first function button 20, the second function button 21, the third function button 22, the directional key 23, the first adjustable eyepiece module 30, the second adjustable eyepiece module 31 and a USB socket can be exposed for the testers to operate. The tester can hold the two sides of the casing 10 with both hands, operate the function buttons and directional key 23 with their fingers, and put the first adjustable eyepiece module 30 and the second adjustable eyepiece module 31 close to both eyes for testing. The schematic diagram in FIG. 2 is a schematic illustration of the internal design of the casing 10 after being exploded along its six sides. It should be emphasized that although the casing 10 in this embodiment is a cuboid, the shape in the present invention is not limited thereto, as long as there are necessary boards.

As mentioned above, the first function button 20, the second function button 21, the third function button 22 and the directional key 23 are installed on the casing 10 through the holes respectively with external portion exposed. Each function button can be given a specific function through the settings of the program. The function buttons are used to input signals to the control module 60, usually to control the hardware. For example, short press (press and then release) the first function button 20 enables portable intelligent multi-function phoropter 1, long press (press and hold for a while) the second function button 21 turns on the liquid crystal display module 50 etc. However, the function buttons can also be assigned the function of operating software. For example, when the third function button 22 is pressed repeatedly, the built-in Tumbling E Chart, Landolt C Chart and other test interfaces will appear on the liquid crystal display module 50 in turn; when the third function button 22 is released from pressing, the current test interface can be used. The directional key 23 includes an up key 231, a down key 232, a left key 233, a right key 234 and an execute key 235 (ok key). This can be used as a tool for executing designed content of software. For example, when the Tumbling E Chart is chosen for the tester for visual inspection, the random “E” (comprising size and opening direction) in the Tumbling E Chart will be displayed on the liquid crystal display module 50. At this moment, the tester needs to use the up key 231, the down key 232, the left key 233 and the right key 234 to answer the opening direction of “E”, so that the control module 60 can judge the tester's vision. When the liquid crystal display module 50 shows “testing completed”, press the execute key 235 to jump out of the Tumbling E Chart test and return to the main menu. It should be emphasized again, types, functions and quantities of the above control elements are not intended to limit the application of the present invention. Physical input hardware that can be programmed based on actual hardware and software operations is within the claimed scope of the present invention.

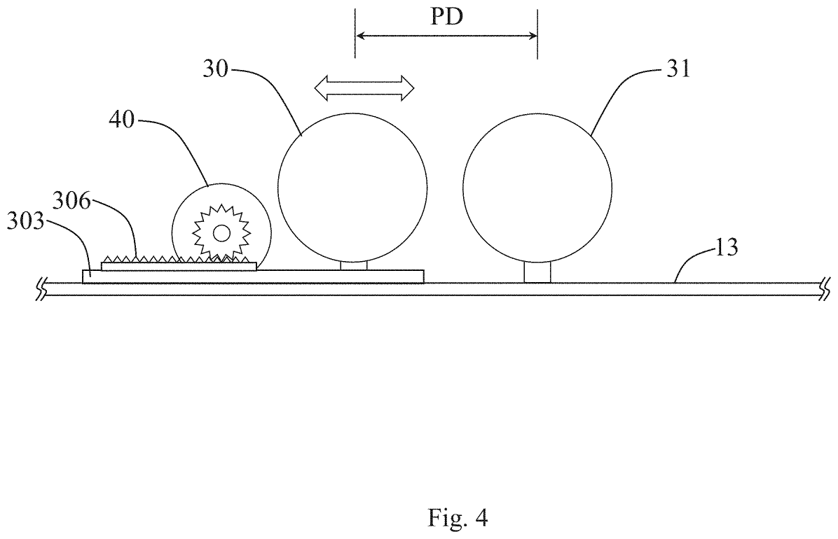

For convenience of description, a side of the casing 10 having 2 adjustable eyepiece modules installed is a first side 11. Opposite the first side 11 is the second side 12. For further explanation, the first adjustable eyepiece module 30 and the second adjustable eyepiece module 31 are movably installed on the first side 11 of the casing 10. Here, “movably” means that the distance between two adjustable eyepiece modules can be adjusted, and the structure of each adjustable eyepiece module can also be adjusted. That is, they can be changed relative to the opening on the casing 10. In order to achieve the above purpose, the structure of the first adjustable eyepiece module 30 is slightly different from that of the second adjustable eyepiece module 31. Since the mechanism parts of the two in FIG. 2 are blocked by the first side 11, for an understanding of the complete structure, please refer to FIG. 3 and FIG. 4. FIG. 3 illustrates and compares structural differences between the first adjustable eyepiece module 30 and the second adjustable eyepiece module 31 in a direction perpendicular to the first side 11. FIG. 4 shows the whole structures of the first adjustable eyepiece module 30 and the second adjustable eyepiece module 31 in a direction parallel to the first side 11.

The top of FIG. 3 shows the first adjustable eyepiece module 30. It mainly includes a first convex lens 301 and a first concave lens 302. The first convex lens 301 and the first concave lens 302 have coincident optical axes. The light beams emitted by the liquid crystal display module 50 are directed to the two lenses. By adjusting the distance between the two lenses, the distance F from the focus point of passed light beams to the first convex lens 301 can be adjusted. In order to achieve the above purpose, the first convex lens 301 is fixed on a first movable board 303 and the first concave lens 302 is fixed on a second movable board 304. The second movable board 304 has a second gear rack 305 installed thereon and is movably placed on the first movable board 303. The second gear rack 305 moves the second movable board 304 parallel to the optical axis of the first concave lens 302 (in the direction of a double arrow on the upper side) by the rotation of a gear mounted to the second micro motor 41. Thereby, the first concave lens 302 will also move in the direction of its optical axis to adjust the distance between the first concave lens 302 and the first convex lens 301. Hence, the distance F will be adjusted as well. It should be emphasized that the positions of the first convex lens 301 and the first concave lens 302 can be interchanged. Namely, the first convex lens 301 can move relative to the first concave lens 302 due to the movement of the second movable board 304. In addition, it can be seen from FIG. 4 that the first movable board 303 has a first gear rack 306 installed thereon. The first gear rack 306 moves the first movable board 303 parallel to a direction vertical to the moving direction of the second movable board 304 (in the direction of the double arrow on the upper side, i.e., parallel to the direction of the first side 11) by the rotation of a gear mounted to the first micro motor 40. Thereby, the distance between the first adjustable eyepiece module 30 and the second adjustable eyepiece module 31, the pupillary distance PD, can be adjusted. Hence, the portable intelligent multi-function phoropter 1 can be used by testers with different pupillary distances. The pupillary distance PD of each tester can also be recorded by the portable intelligent multi-function phoropter 1.

The bottom of FIG. 3 shows the second adjustable eyepiece module 31. It includes a second convex lens 311 and a second concave lens 312. The second convex lens 311 and the second concave lens 312 also have coincident optical axes. The light beams emitted by the liquid crystal display module 50 are directed to the two lenses. By adjusting the distance between the two lenses, the distance F′ from the focus point of passed light beams to the second convex lens 311 can be adjusted. In order to achieve the above purpose, second convex lens 311 is fixed on side (bottom side 13) of the casing 10 and inside the casing 10, and the second concave lens 312 is fixed on a third movable board 313. The third movable board 313 has a third gear rack 314 installed on and is movably placed on the bottom side 13 of the casing 10 and inside the casing 10. The third gear rack 314 moves the third movable board 313 parallel to the optical axis of the second concave lens 312 (in the direction of a double arrow on the bottom side) by the rotation of a gear mounted to the third micro motor 42. Thereby, the second concave lens 312 also moves in the direction of its optical axis to adjust the distance between the second convex lens 311 and the second concave lens 312. Hence, the distance F′ will also be adjusted. Similarly, the positions of the second convex lens 311 and the second concave lens 312 can be interchanged. Namely, the second convex lens 311 can move relative to the second concave lens 312 due to the movement of the third movable board 313.

In the present embodiment, the first adjustable eyepiece module 30 is used for the left eye and the second adjustable eyepiece module 31 is used for the right eye. In practice, the two can be interchanged. When testing, since the test image sent by the liquid crystal display module 50 will go through the first adjustable eyepiece module 30 and the second adjustable eyepiece module 31 at the same time, it can be seen by the left eye and the right eye, respectively. Therefore, the tester can close one eye and let the other eye perform the test. In addition, the distance F and distance F′ shown on FIG. 3 are different. Namely, a test image can allow different eyes to see different degrees (depending on the distance between the focal point and the retina of the human eye), which can be achieved by adjusting the second micro motor 41 and the third micro motor 42. Therefore, by designing the adjustable font size on the liquid crystal display module 50 and fine-tuning the distance between the convex lens and the concave lens, the first adjustable eyepiece module 30 and the second adjustable eyepiece module 31 can change the focus location for light beams emitting from the inside of the casing 10 to the outside, further to simulate an actual distance of 20 feet to 50 feet required for eye optometry into a distance of about 10 cm, as a usual vision test. In summary, each of the first micro motor 40, the second micro motor 41 and the third micro motor 42 can interact with one of the two adjustable eyepiece module and drives to adjust the corresponding adjustable eyepiece module, so as to change the focus location for light beams emitting from the inside of the casing 10 to the outside and a spacing between the two adjustable eyepiece modules. The actions of these micro motors can be controlled by the control module 60. Preferably, these micro motors are stepper motors, which can precisely control the amount of rotation.

The liquid crystal display module 50 is installed on the second side 12 of the casing 10 and inside the casing 10, projecting images to the direction of the two adjustable eyepiece modules.

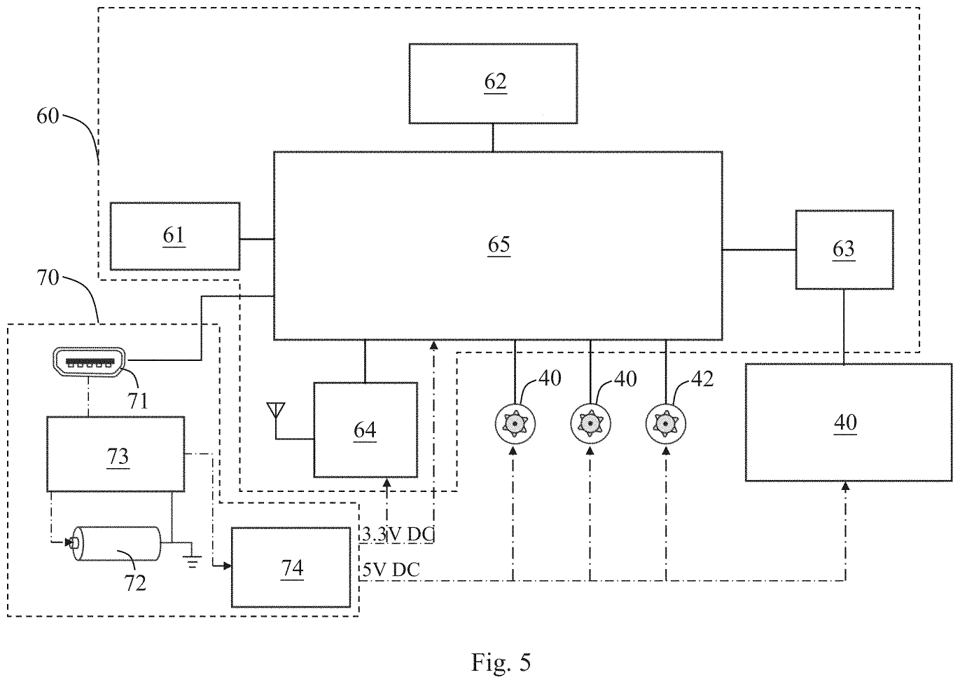

The control module 60 is installed in the casing 10 and signally connected to the control elements, the micro motors and the liquid crystal display module 50. To illustrate how these components are connected and how they interact, see FIG. 5. FIG. 5 is a block diagram of the liquid crystal display module 50, the control module 60 (shown by a dotted line frame), the power supply module 70 (shown by a dotted line frame) and micro motors. It can be seen from FIG. 5 that the control module 60 comprises a flash memory module 61, a synchronous dynamic random access memory module 62, a graphics processor 63, a wireless communication module 64 and a controller 65. The flash memory module 61 is used to store a test result which will be mentioned below for a long time, and codes and code-related data operated in the controller 65. The latter may be the test pattern for optometry. The synchronous dynamic random access memory module 62 is used to temporarily store some programs and data that will be used by controller 65. Once the power is not turned on, the data stored in it will be lost. The graphics processor 63 and the liquid crystal display module 50 are signally connected to control the image presented by the liquid crystal display module 50. The wireless communication module 64 is used for wireless signal connection with a receiving device (not shown) outside the portable intelligent multi-function phoropter 1 to transmit the test result to the receiving device. The test result is shown on the receiving device for performing subsequent analysis tasks. The wireless communication module 50 may be a Bluetooth module or a Wi-Fi module. It is also possible to have both at the same time.

The controller 65 is a core element for operating the portable intelligent multi-function phoropter 1. The controller 65 is signally connected (connection between the components is represented by the solid line in FIG. 5) to the control elements, a USB socket 71 of the power supply module 70, the synchronous dynamic random access memory module 62, the flash memory module 61, the graphics processor 63, the wireless communication module 64 and the micro motors, and electrically connected to a power management chip 74 in the power supply module 70. The controller 65 executes the default program to execute specified operations, temporarily stores part of the codes and called code-related data in the synchronous dynamic random access memory module 62, and transmits the stored test result to a receiving device wirelessly connected through the wireless communication module 50. The controller 65 can be a programmable logic device chip, which can cooperate with a specific operating program to execute an application program (based on code) or directly execute a specific application program. Therefore, the control module 60 may be regarded as an embedded computer in the portable intelligent multi-function phoropter 1. The control module 60 also comprises other active components, passive components, control chips, etc., mounted on a circuit board. This technique is well known to those with specialized knowledge in the field, so it will not be described in detail.

As mentioned above, the controller 65 (control module 60) can perform specified operations according to the application program it executes. These operations comprises: a) making the liquid crystal display module 50 present an operation interface; b) executing a default program according to the content of the operation interface and receiving control signals from the control elements to decide execute commands; c) making the liquid crystal display module 50 present a designated test pattern according to a first execute command; d) driving the micro motors according to a second execute command; e) determining the test result according to a third execute command; and f) recording the test result and outputting the test result according to a fourth execute command. These operations are described below.

In the operation a), the operation interface is an interface that guides the tester to perform the vision test with a series of text, graphics and tables, and changes the content accordingly. The process is provided according to the design of the application, and it is operated with the control elements. In the operation b), the execute command is the control signals (such as long press, short press) of the specific control element, which is used in conjunction with the display data of the operation interface to feed back the instructions for the application to execute the next step. For the convenience of description, each execute command proposed below, such as the first execute command, the second execute command, etc., is only the more important one among many execute commands, and is the focus of the present invention.

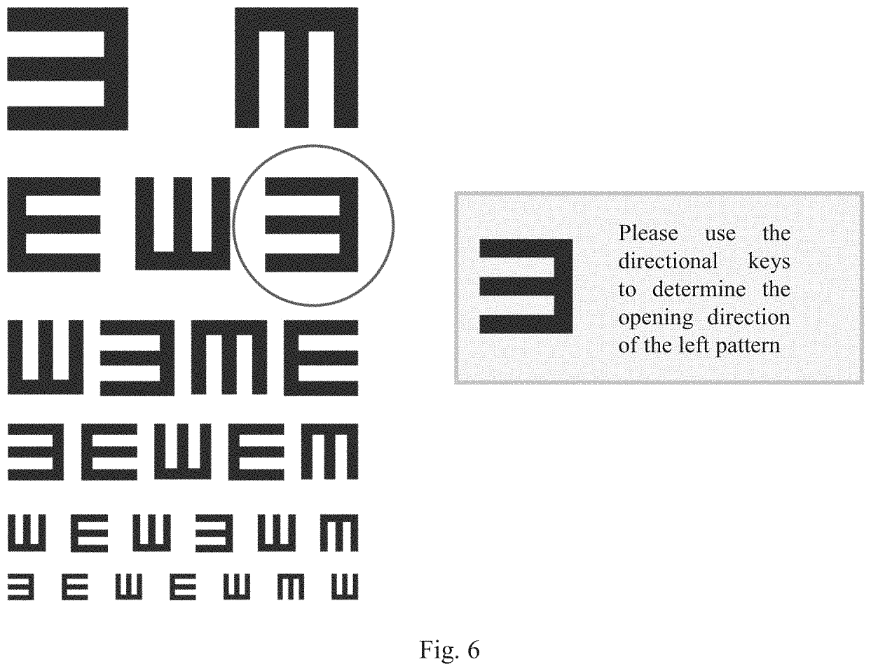

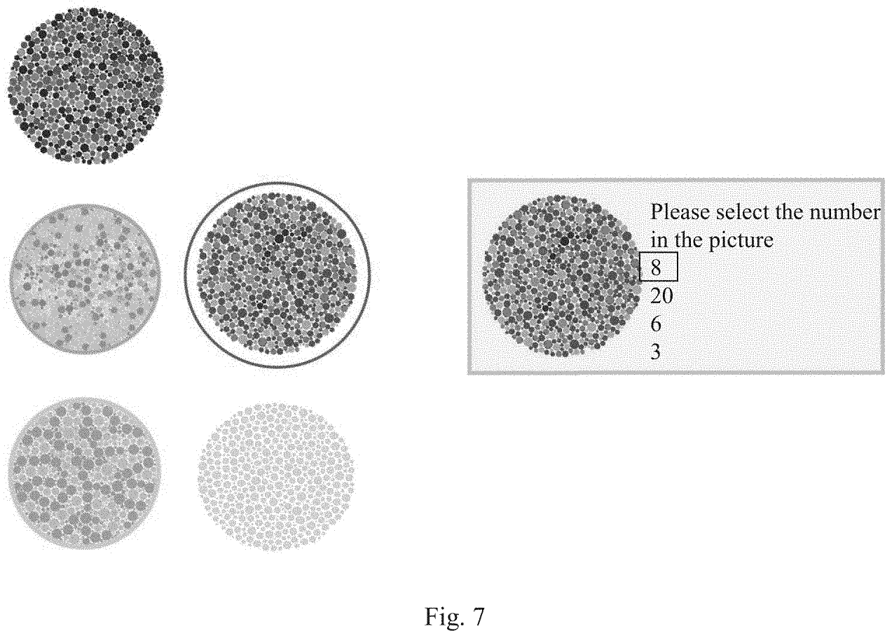

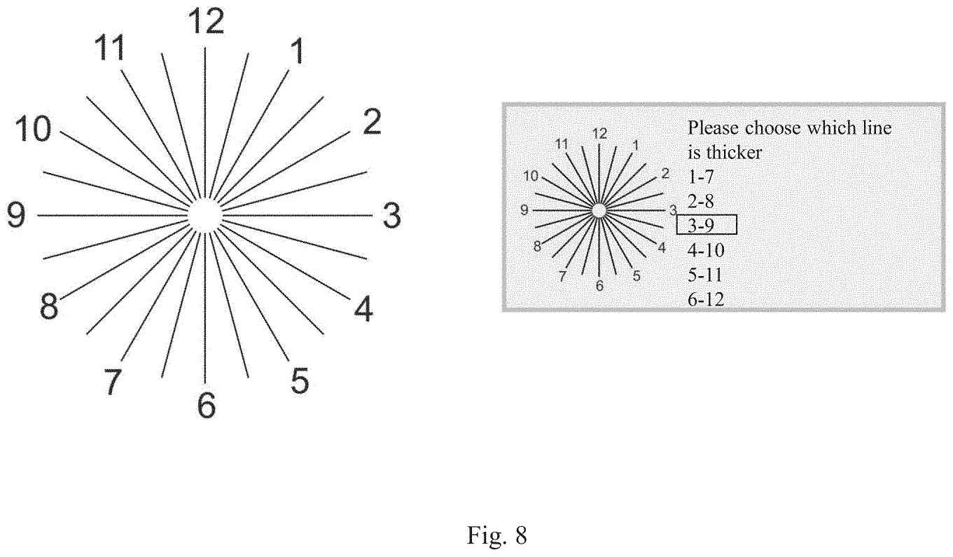

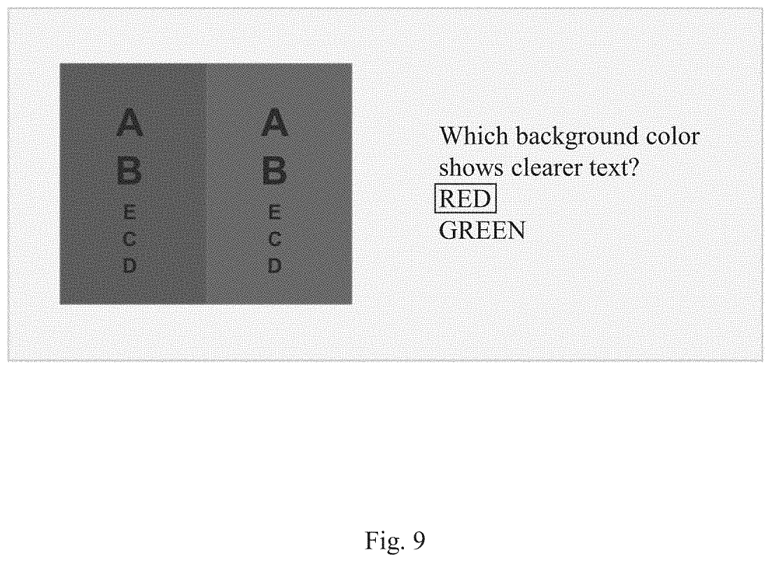

In the operation c), the designated test pattern that the first execute command asks the liquid crystal display module 50 refers to complete or part of Tumbling E Chart, Landolt C Chart, Color Blindness Test pattern, Astigmatism Test pattern or Duochrome Test pattern. For a better understanding of this, see FIG. 6 to FIG. 9. FIG. 6 shows the Tumbling E Chart and a portion of the Tumbling E Chart presented on the liquid crystal display module 50. FIG. 7 shows the Color Blindness Test pattern and a portion of the Color Blindness Test pattern presented on the liquid crystal display module 50. FIG. 8 shows a complete Astigmatism Test pattern and a diagram presented on the liquid crystal display module 50. FIG. 9 shows a type of Duochrome Test pattern presented on the liquid crystal display module 50. In FIG. 6, the left side shows the complete Tumbling E Chart. The application can randomly select an “E” pattern to show on the liquid crystal display module 50, and display the operation instructions on the right of the “E” pattern. The tester can follow the instructions and use the directional key 23 to adjust the box to select the “answer”. The application will use the answer to determine the test result, such as yes or no to pass the pattern test, what the vision test value is, etc. Landolt C Chart is operated in the same way, and will not be repeated here. In FIG. 7, the complete Color Blindness Test pattern is shown on the left. The application can randomly select a pattern to show it on the liquid crystal display module 50, and display the operation instructions on the right of the pattern. The tester can follow the instructions and use the directional key 23 to adjust the box to select the number in the pattern. The application will use the selection to determine the test result, such as “Deuteranopia,” “Protanopia” or “Tritanopia.” In FIG. 8, the complete Astigmatism Test pattern is shown on the left. The application can present the Astigmatism Test pattern on the liquid crystal display module 50. Through the adjustment of the adjustable eyepiece module, the Astigmatism Test pattern has a different sense of line variation for the tester, and then the tester can choose the thicker line as explained on the right. The application can use this selection to determine the test result, such as the degree of astigmatism. The thickness of the line can also be adjusted through the control element. In FIG. 9, the application presents a two-color test pattern with a red and green background and different text combinations, determining the reception status of the pattern by the human eye. And this is also the test result of the application. Operationally, if the letters in the red block look sharper, press the up key 231 to simulate −0.25 DS until two sides are balanced; or press the down key 232 to simulate +0.25 DS until both sides are balanced. It should be emphasized that the aforementioned display modes are only a part of the various aspects of the present invention, and do not limit the present invention.

In addition, a technical feature of the present invention is that the test pattern may further be a simulated image of a specific object seen through a spectacle lens with a specific eyeglass prescription. The present invention has built-in hundreds of image-processed patterns to simulate and represent the images seen after passing through lenses of different eyeglass prescriptions. When one of the images is displayed on the liquid crystal display module 50 and projected onto the tester's eyes through the adjustable eyepiece modules, the tester sees it as if he had put on a lens of the corresponding degree. For example, an image that simulates 100-degree myopia and the tester sees it as if wearing a 100-degree myopia lens. Hyperopia does the same way. Furthermore, simultaneously presenting the simulated images of the specific object each seen through a spectacle lens with a specific eyeglass prescription can simulate the specific object seen through the spectacle lenses superimposed. For example, the simultaneous display of an image of an apple through a 100-degree spectacle lens and an image of an apple through a 50-degree spectacle lens means that the tester sees the image of an apple through a 150-degree spectacle lens. It seems to be a superposition of spectacle lenses of different eyeglass prescriptions.

The operation d) is that the portable intelligent multi-function phoropter 1 adjusts the adjustable eyepiece modules to change the focus location when light beams are emitted from the inside of the casing 10 to the outside according to the application's command to let tester comes to different test environments. Of course, In order to allow different testers to see the information of the operation interface clearly, testers can also manually adjust the adjustable eyepiece module to be free from the limitation of the application.

The operation e) is a follow-up after the operation c) is processed and determines the aforementioned test result. Subdivide the operation e) and it has the following sub-steps. First, record the control element pressed by the user and an operation mode of the pressed control element according to the complete or the part of chart or pattern presented. What this sub-step needs to do is to record the decision made by the tester under the condition that the information is displayed by the liquid crystal display module 50. Secondly, compare the complete or the part of chart or pattern presented, the control element pressed and the operation mode of the control element pressed with a database storing complete or each part of the chart or pattern, corresponding control elements, the operation modes of the corresponding control elements, and corresponding eye status descriptions. The database is built in the flash memory module 61. For example, the database records the opening direction of each E figure in the Tumbling E Chart, the control element that needs to be operated, and correct and wrong operation modes of the control elements corresponding to the opening direction (e.g., for the pattern “E”, the up key 231 is wrong, the down key 232 is wrong, the left key 233 is wrong and right key 234 is correct). Eye status description is whether it meets the vision requirements of the pattern, for example, vision is 0.8. Thirdly, select the eye status description corresponding to the exact comparison result as the test result. Due to the correspondence of the database, the test result of the tester can be quickly detected.

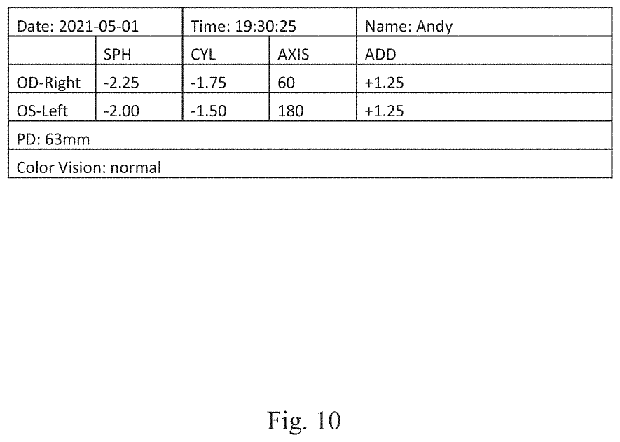

The operation f) is to record the test result determined by the operation e). The way the fourth execute command outputs the test result can be diverse. In addition to the transmission to the receiving device through the wireless communication module 64, it can also be transmitted to the device connected by a communication line through the USB socket 71. The aspect of the output test result is also diverse, for example, the table of test results shown in FIG. 10. In the table of test results, there may be set date, time, name, Wi-Fi, Bluetooth and email address in a setting form. Then, the table of test results can be sent out through the email address.

The power supply module 70 is installed in the casing 10 and electrically connected to (connection between the components is represented by the dashed line in FIG. 5) the micro motors, the liquid crystal display module 50 and the control module 60, to provide power required for operation to the electrically connected components. The power supply module 70 may further comprises: the USB socket 71, a secondary battery 72, a charge and discharge control chip 73 and a power management chip 74. The USB socket 71 can receive external power and transmit it inside the casing 10. In addition, the USB socket 71 can transmit data, such as updating the code of the application, new test patterns, etc., from the outside to the controller 65 due to its specifications, or transfer the table of test results from controller 65 to the external device. The secondary battery 72, such as a lithium battery, is used as a power storing device of the portable intelligent multi-function phoropter 1 and controlled by the charge and discharge control chip 73. The charge and discharge control chip 73 is electrically connected to the USB socket 72 and the secondary battery 72. It stores the external power received by the USB socket 71 to the secondary battery 72, and outputs the power in the secondary battery 72 to the power management chip 74. The power management chip 74 can transform the voltage of the power outputted from the charge and discharge control chip 73 to a number of voltage values, such as 3.3V or 5V, providing to electrically connected elements, respectively.

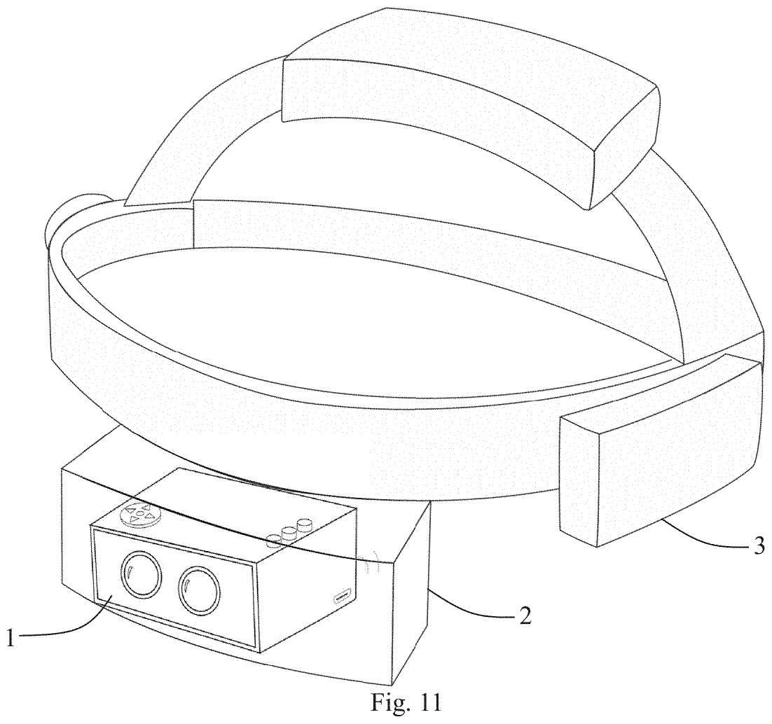

In application, in addition to independent use, the portable intelligent multi-function phoropter 1 can be installed on a platform 2 as illustrated in FIG. 11 The platform 2 is further connected to a headgear 3. It is convenient for the tester to wear and use.

In summary, the present invention has the following advantages. The present invention reduces the traditional large-volume fixed eye examination instrument into a hand-held device that is easy to carry, but still retains complete functions. The present invention makes full use of the powerful computing power of the computer chip, and can instantly calculate the complex mathematical and optical parameters required for operation, as well as the results required for graphics. The large-capacity storage memory can store various patterns and texts required for existing eye optometry. The present invention uses a graphics processor with high efficiency drawing and display capability and a high-resolution liquid crystal display module, which can display various graphics, text and real-time operation instructions. The invention adopts the combination of a concave lens and a convex lens with the adjustable size display font and the calculation of computer software to simulate an actual distance of 20 feet to 50 feet required for eye optometry into a distance of about 10 cm, fitting the small size of this device. In addition, the invention uses computer graphics processing and special software algorithm to simulate the graphics and texts seen by the tester. Therefore, the user sees the graphics as if he saw the effect through spectacle lenses with different eyeglass prescriptions and different angles of astigmatism lenses. Hundreds of charts or patterns are pre-stored in the flash memory module, which can be regarded as virtual lenses and called to use when testing. Multiple virtual lenses can be superimposed. Different eyeglass prescriptions and astigmatism lenses used in traditional optometry will be converted into different graphics by the application software with its intelligent algorithm. After the testing, with the immediate operation of the controller and the comparison with the data in the current database data, the accurate visual acuity result can be obtained. Through the adjustable eyepiece modules with adjustable spacing and the focus calculation of the screen grid lines, the pupil distance of two eyes can be measured.

Different from the existing phoropters, the present invention can be widely used in the following: 1, personal and household use; 2, basic vision examination in the school medical center; 3, remote areas or villages without sufficient resources or equipment for vision test; and 4, an aid or auxiliary device in a clinic or hospital.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims, which are to be accorded with the broadest interpretation so as to encompass all such modifications and similar structures.

What is claimed is:

1. A portable intelligent multi-function phoropter, comprising:
a casing;
a plurality of control elements, installed on the casing with external portion exposed;
two adjustable eyepiece modules, movably installed on a first side of the casing;
a plurality of micro motors, wherein each micro motor interacts with one of the adjustable eyepiece modules and drives to adjust the corresponding adjustable eyepiece module, so as to change the focus location for light beams emitting from the inside of the casing to the outside and a spacing between two adjustable eyepiece modules;
a liquid crystal display module, installed on a second side opposite the first side of the casing and inside the casing, projecting images to the direction of the two adjustable eyepiece modules;
a control module, installed in the casing and signally connected to the control elements, the micro motors and the liquid crystal display module, executing following operations:
making the liquid crystal display module present an operation interface;
executing a default program according to the content of the operation interface and receiving control signals from the control elements to decide execute commands;
making the liquid crystal display module present a designated test pattern according to a first execute command;
driving the micro motors according to a second execute command;
determining a test result according to a third execute command; and
recording the test result and outputting the test result according to a fourth execute command; and
a power supply module, installed in the casing and electrically connected to the micro motors, the liquid crystal display module and the control module, providing power required for operation to the electrically connected components.

2. The portable intelligent multi-function phoropter according to claim 1, wherein the control elements further comprise at least one function button and a directional key to input signals to the control module.

3. The portable intelligent multi-function phoropter according to claim 1, wherein a first adjustable eyepiece module comprises:
a first convex lens, fixed on a first movable board; and
a first concave lens, fixed on a second movable board, wherein the second movable board has a second gear rack installed thereon and is movably placed on the first movable board, and the second gear rack moves the second movable board parallel to an optical axis of the first concave lens by the rotation of a gear mounted to one of the micro motors,
wherein the first movable board has a first gear rack installed thereon, and the first gear rack moves the first movable board parallel to a direction vertical to the moving direction of the second movable board by the rotation of a gear mounted to one of the micro motors.

4. The portable intelligent multi-function phoropter according to claim 1, wherein a second adjustable eyepiece module comprises:
a second convex lens, fixed on one side of the casing and inside the casing; and
a second concave lens, fixed on a third movable board, wherein the third movable board has a third gear rack installed thereon and is movably placed on the side of the casing and inside the casing, and the third gear rack moves the third movable board parallel to an optical axis of the second concave lens by the rotation of a gear mounted to one of the micro motors.

5. The portable intelligent multi-function phoropter according to claim 1, wherein the power supply module further comprises:
a USB socket, receiving external power and transmitting information;
a secondary battery;
a charge and discharge control chip, electrically connected to the USB socket and the secondary battery, storing the external power received by the USB socket to the secondary battery, and outputting the power in the secondary battery; and
a power management chip, transforming the voltage of the power outputted from the charge and discharge control chip to a plurality of voltage values, providing to electrically connected elements, respectively.

6. The portable intelligent multi-function phoropter according to claim 5, wherein the control module further comprises:
a flash memory module, storing the test result, codes and code-related data;
a synchronous dynamic random access memory module;
a graphics processor, signally connected to the liquid crystal display module to control the image presented by the liquid crystal display module;
a wireless communication module; and
a controller, signally connected to the control elements, the USB socket, the synchronous dynamic random access memory module, the flash memory module, the graphics processor, the wireless communication module and the micro motors and electrically connected to the power management chip, executing the default program to execute the operations, temporarily storing part of the codes and called code-related data in the synchronous dynamic random access memory module, and transmitting the stored test result to a receiving device wirelessly connected through the wireless communication module.

7. The portable intelligent multi-function phoropter according to claim 6, wherein the wireless communication module is a Bluetooth module or a Wi-Fi module.

8. The portable intelligent multi-function phoropter according to claim 1, wherein the test pattern is complete or part of Tumbling E Chart, Landolt C Chart, Color Blindness Test pattern, Astigmatism Test pattern or Duochrome Test pattern.

9. The portable intelligent multi-function phoropter according to claim 8, wherein steps for the third execute command to determine the test result comprises:
recording the control element pressed by the user and an operation mode thereof according to the complete or the part of chart or pattern presented;
comparing the complete or the part of chart or pattern presented, the control element pressed and the operation mode of the control element pressed with a database storing complete or each part of the chart or pattern, corresponding control elements, the operation modes of the corresponding control elements, and corresponding eye status descriptions; and
selecting the eye status description corresponding to the exact comparison result as the test result.

10. The portable intelligent multi-function phoropter according to claim 1, wherein the adjustable eyepiece module changes the focus location for light beams emitting from the inside of the casing to the outside, simulating an actual distance of 20 feet to 50 feet required for eye optometry into a distance of about 10 cm.

11. The portable intelligent multi-function phoropter according to claim 1, wherein the test pattern is a simulated image of a specific object seen through a spectacle lens with a specific eyeglass prescription.

12. The portable intelligent multi-function phoropter according to claim 11, wherein simultaneously presenting the simulated images of the specific object each seen through a spectacle lens with a specific eyeglass prescription simulates the specific object seen through the spectacle lenses superimposed.
